(19) United States
(12) Patent Application Publication
(10) Pub. No.: US 2006/0186219 A1
(43) Pub. Date: Aug. 24, 2006

(54) Solar evaporated air freshener

(76) Inventor: Carl Ernest Kent, Eden Prairie, MN (US)

Correspondence Address: MERCHANT & GOULD PC, P.O. BOX 2903, MINNEAPOLIS, MN, 55402-0903, US

(21) Appl. No.: 11/329,806

(22) Filed: Jan. 10, 2006

Related U.S. Application Data
(60) Provisional application No. 60/655,849, filed on Feb. 24, 2005.

(51) Int. Cl. A24F 25/00; A61L 9/04

(52) U.S. Cl. 239/60; 239/34

(57) ABSTRACT

An air freshener apparatus delivering passive solar collection, solar energy transfer, evaporation of a plurality of solids, gels, and liquids, vapor diffusion process, and working in conjunction with the solar filtering characteristics provided by a plurality of standard glass panes contained in automobiles an buildings. The apparatus includes a panel for absorbing solar energy, a process for coating said panel with a plurality of highly solar heat absorptive coating materials and optional photo-chromic coatings, a plurality of breathable insulating substrate membranes directly adjacent to said coated panel, a sealant, and an impregnated plurality of microencapsulated and non-microencapsulated solids, gels, or liquids into said plurality of breathable insulating substrate membranes. Initial solar collection and subsequent evaporation of said plurality of microencapsulated and non-microencapsulated solids, gels or liquids into vapors is achieved by means of placing the apparatus to the inner side of a plurality of standard glass panes in automobiles and buildings. Apart from aiding in the evaporation of said plurality of microencapsulated and non-microencapsulated solids, gels or liquids into vapors, solar energy transfer from said plurality of solar coatings on apparatus surface to a plurality of breathable insulating membranes is used to achieve diffusion of said vapors and associated fragranced aromas by means of natural air flow in one embodiment of hanging the air freshener apparatus. In a second embodiment, diffusion of said vapors and associated fragranced aromas is achieved by means of connecting the present invention, with its attachable clip, to the louvers of an automobile's ventilation system that, when blowing air, diffuses fragrance into the air inside of an automobile. In a novel feature, one embodiment of the invention discloses physical space on the surface of the solar evaporated air freshener to absorb a plurality of standard inks and inks containing light sensitive, color changing photo-chromic molecules with said surface space being used for brand, product and or service advertising.

### RELATED APPLICATION(S)

This application claims priority to U.S. Provisional Application No. 60/655,849, filed Feb. 24, 2005. Application No. 60/655,849 is incorporated herein by reference in its entirety.

### FEDERALLY SPONSORED RESEARCH OR DEVELOPMENT

Not applicable.

### SEQUENCE LISTING OF TABLES OR COMPUTER PROGRAMS OR COMPACT DISC

Not applicable.

### BACKGROUND OF THE INVENTION

1. Technical Field

This invention relates to a novel solar energy apparatus and more particularly relates to a new utility process and apparatus for absorbing, storing and transferring solar energy for use in heat-induced evaporation and of fragranced gels, solids or liquids impregnated in breathable insulating membranes contained within the apparatus, and subsequent distribution of resulting fragranced vapors.

Microencapsulated and non-micro-encapsulated solids, gels and liquids, such as essential oils (or those oils extracted from organic plant matter as opposed to synthetically manufactured oils), are key ingredients to a growing wave of for-sale aroma products that are designed as therapeutic air enhancers, some with medicinal value to help users reduce lung congestion, breathe easier, and purify their environment of malodors. As “aroma-therapy” is a nascent-growing market segment with strong consumer demand for a host of products in both the United States and foreign countries, there is great attention being given to alternative diffusion methods. The method of the present invention is especially appealing because it is based on using solar energy, an unlimited, renewable energy source with the absence of pollution and other ecological problems therewith. The present invention relates to the freshening or fragrancing of the air in an enclosed area by using devices and methods for distributing such a fragrance or freshener.

A number of devices and methods for air freshening or fragrancing have been available for some time. Common devices include aerosol sprays, scented candles, impregnated waxes, impregnated polymers, and various electrical and mechanical devices which draw a fragrance out of a storage device of some sort and then distribute it throughout a room. Although advantageous in some respects, many of these devices are unsuitable for certain applications. Aerosol sprays require active distribution by a user in periodic fashion while candles use open flames which must be carefully attended. Several of the impregnated products depend on evaporation of their solids, gels and liquids by means of sublimation or gradual evaporation by means of the temperature and humidity of the surrounding environment. The newer mechanical-electrical gadgets are quite interesting, but many are relatively expensive and are most suitable to professional applications or for those willing to indulge in expensive gadgetry or the use of non-renewable energy. More specific attempts have focused on the use of containers placed adjacent to light bulbs so that the heat from the bulb encourages a liquid or solid fragrant material in the container to evaporate and scent a room.

The disadvantage of most of these devices is that the liquid fragrant material must be carefully added while the container is adjacent the bulb. Alternatively, if the container is filled with liquid away from the bulb, the filled container must be carefully transferred to and positioned on the bulb in order to avoid spilling the contents. Other such devices must be “loaded” by literally soaking them in fragrant materials and more than a few of the devices are relatively complicated in view of the rather straightforward effect desired.

2. Prior Art References

Suggested Current U.S. Class: 126/618; 422/124, 126/631; 126/704

Suggested International Class: F24J 003/02

Field of Search: 126/417, 419, 428, 429, 424, 425, DIG. 1, 430, 442, 448, 436, 450

References Cited [Referenced By]
U.S. Patent Documents

| | | | |
|---|---|---|---|
| 3711023 | June, 1973 | Smith | 239/54. |
| 3832992 | September, 1974 | Trombe et al | 126/270. |
| 3863621 | February, 1975 | Schoenfelder | 126/270. |
| 3930796 | January, 1976 | Haensel | 422/180. |
| 3990848 | November, 1976 | Corris | 422/49. |
| 4002159 | January, 1977 | Angilletta | 126/429. |
| 4324226 | September, 1977 | Cook | 126/632. |
| 4068652 | January, 1978 | Worthington | 126/603. |
| 4074111 | February, 1978 | Hunter | 422/125. |
| 4100914 | July, 1978 | Williams | 126/429. |
| 4324226 | April, 1982 | Beck | 126/430. |
| 4324804 | January, 1984 | Lee | 126/572. |
| 4579717 | April, 1986 | Gyulay | 422/125. |
| 4647428 | March, 1987 | Gyulay | 422/4. |
| 4808347 | February, 1989 | Dawn | 261/30. |
| 5342584 | August, 1994 | Fritz et al. | 422/124. |
| 5422078 | June, 1995 | Colon | 422/123 |
| 5938076 | August, 1999 | Ganzeboom | 222/23. |
| 6197263 | March, 2001 | Blount | 422/125. |
| 6258170 | July, 2001 | Somekh | 118/715. |
| 6264887 | July, 2001 | Farmer | 422/5 |
| 6632405 | October, 2003 | Lua | 422/124. |

Prior Art-Air Fresheners

It can be appreciated that air fresheners have been in use for years. Typically, air fresheners are comprised of U.S. Pat. No. 5,342,584, an air freshener device and cartridge with battery as means of power. U.S. Pat. No. 3,711,023 (Smith), and U.S. Pat. No. 6,197,263 (Blount) air freshener devices utilizing an air conditioning system which releases the evaporated volatile substance into the air to produce an odor. U.S. Pat. No. 3,990,848, an air flow induction device air freshening apparatus with a fan mounted in the housing. A fan driven by a motor is connected with the battery. U.S. Pat. Nos. 4,647,428; 4,579,717; 4,407,411; and 3,930,796 issued to Gyulay, Gyulay, Hunter, and Haensel respectively are all room air freshening devices that use a light bulb for vaporization of liquid. They work when the light bulb is turned on to heat and vaporize fragrance to permeate the room. The problem with these elastomeric O rings is that these room freshener devices utilize a light bulb that is plugged into the electrical system. The light bulb has to be turned on and at the same time consumes standard, non-renewable electricity to function.

U.S. Pat. No. 6,264,887 issued to Farmer is an air freshening device comprised of a polymer shaped in the form of a clothes-pin with a clip containing apertures impregnated with gel fragrance; the apparatus is attached by means of its clip to the louvers of an automobile ventilation system. A problem with conventional motor-enabled air fresheners are that, as in U.S. Pat. Nos. 6,264,887 and 3,711,023, the devices solely rely on use air conditioning systems (of either automobiles as in the case of U.S. Pat. No. 6,264,887, or automobiles and buildings as in the case of U.S. Pat. No. 3,711,023) to release the evaporated volatile substance into the air to produce an odor. The utilization of air conditioning systems makes these devices depended on having the air conditioning systems working. Further, with U.S. Pat. No. 6,264,887, the use of a clip to attach to the louvers limits this apparatus to only being functional in automobile ventilation systems for there is no other means of diffusion, whereas in the present invention, the use of a clip to attach the apparatus to the louvers of an automobile is merely an option to aid in diffusion, given that the present invention's use of solar heat to rapidly advance evaporation also aids in apparatus based diffusion of fragrance.

U.S. Pat. No. 5,422,078 issued to Colon discloses a method and a means to diffuse fragrance into a compartment by means of an apparatus consisting of a reusable holder and replaceable scented polymer cartridges, with the apparatus being clipped to the louvers of the air vents of automobiles, or buildings. Colon makes mention of auxiliary use of a solar powered fan to diffuse fragrance. In significant departures from the present invention, Colon makes no mention of using solar energy for the purpose of generating heat-induced evaporation of fragranced liquids, which in the case of Colon, are injected into a polymer substrate. Instead, to deliver fragrance diffusion, Colon relies on gradual evaporation of a polymer substrate based on surrounding room or automobile vent system temperatures and humidity. Also in difference from the present invention, Colon exclusively uses scented polymer materials as the substrate for impregnated fragrances.

U.S. Pat. No. 6,197,263, Blount, discloses a method and means to diffuse fragranced materials through optional use of solar energy collection from a photovoltaic cell and storage of said solar energy in a battery for warming of a fragrance cartridge. As indicated in the embodiment of Blount's invention, for the apparatus to function, the fragrance cartridge housing must be attached to an automobile's vent system, thereby removing the possibility of the apparatus serving as a stand-alone air freshener for rooms, unlike the present invention which can be used in rooms and automobiles. Also, Blount uses a separate component scent cartridge, whereas the present invention needs no cartridges as sent is direct applied in the insulating substrate, thereby saving money and providing ease of use in that when the device reaches the end of its useful life, the consumer simply replaces the entire apparatus, which is made of recyclable content. In addition, as opposed to a photovoltaic cell, the present invention utilizes a system of solar coatings applied to the apparatus surface, glass pane (in the room or automobile) and an insulated substrate, again with a substantially less expensive cost than Blount and providing ease of use in that when the device reaches the end of its useful life, the consumer simply replaces the entire apparatus, which is made of recyclable content.

U.S. Pat. No. 6,632,405, Lua, discloses a method and means to diffuse fragranced materials through use of solar energy collection from a photovoltaic cell and storage of said solar energy in a battery for the sole use of transferring energy to operate an oscillating fan to diffuse fragranced materials. Unlike the present invention, Lua makes no mention of, and has no apparatus for, converting collecting, collecting, and converting solar energy for the purpose of generating radiated heat for use in effecting evaporation of fragranced materials, liquid or solid.

The main problem with conventional air fresheners are that, as in U.S. Pat. Nos. 5,342,584 and 3,990,848, is that the devices need the usage of batteries or Alternating Current (A/C) from an electrical outlet. The utilization of batteries to power the devices need more attention to the operation of the devices, need the constant change of batteries, add more cost to the device because of the cost of batteries. Another problem with existing products is that, as in U.S. Pat. No. 4,808,347, the air freshening device is plugged into a standard device as in this case the cigarette lighter socket in a vehicle. Another problem with existing products is that, as in U.S. Pat. No. 4,808,347, the air freshening device is plugged into a standard cigarette lighter socket in a vehicle. Again, the device is dependent on another device as in this case the cigarette lighter, which on occasion may not be functioning.

While these devices may be suitable for the particular purpose to which they address, they are not as suitable for providing liquid evaporation as a device with solar-powered evaporation and diffusion utilizing the energy derived from natural sunlight or light emitted from regular room light fixture. The solar-power battery is operationally connected to an oscillating device wherein a fan is connected. The oscillating or to and fro movement of the fan moves fragrance emitted from the volatile substance of the fragrance out of the housing to the room or vehicle being freshened. The main problem with conventional motor enabled air fresheners are that, as in U.S. Pat. Nos. 5,342,584 and 3,990,848, the devices need the usage of batteries. The utilization of batteries to power the devices need more attention to the operation of the devices, need the constant change of batteries, add more cost to the device because of the cost of batteries.

Another problem is that, as in U.S. Pat. No. 3,711,023, the device uses the air conditioning system to release the evaporated volatile substance into the air to produce an odor. The utilization of the air conditioning system makes the device depended on the time the air conditioning system is working. Also, another problem is that, as in U.S. Pat. No. 4,647,428, the room freshener device utilizes a light bulb that is plugged to the electrical system. The light bulb has to be turned on and at the same time consume electricity to function. Another problem with existing products is that, as in U.S. Pat. No. 4,808,347, the air freshening device is plugged into a standard device as in this case the cigarette lighter socket in a vehicle. Another problem with existing products is that, as in U.S. Pat. No. 4,808,347, the air freshening device is plugged into a standard cigarette lighter socket in a vehicle. Again, the device is dependent on another device as in this case the cigarette lighter, which on occasion may not be functioning.

Prior Art-Solar Devices

Other types of solar energy collection, storage and transferring devices abound. Solar energy has been used for heating air by a number of investigators. It is well recognized that an enclosure having a transparent window facing the sun will become warm inside. Of particular interest with respect to solar energy devices are those apparatuses which may be efficiently operated yet simple to construct, inexpensive, and easy to maintain. Of particular interest with respect to the consuming public are those solar energy converting devices which may be fabricated in small units, mass produced, allowing use by individual consumers.

In the winter time, the degree of warmth will be greater for (1) black, or absorbing surfaces, (2) insulated sides and back, (3) maximum transparency for solar radiation, (4) maximum opacity for re-radiation from the black surface back through the window, (5) minimum emissivity of the absorber for infra-red, or heat wavelengths, and (6) minimum heat transmission by conduction and convection from the heated surfaces through the window.

Usable systems are possible even when the emissivity for low temperature radiation is as high as the absorptivity for solar energy because of the low emitting temperature of the black surface compared with the temperature of the sun. Therefore, spectrally selective coatings and windows are excellent refinements for optimizing performance.

Much good work has been done in combining the properties of a transmitting window with those of an opaque insulating wall. The best methods use an unshaded, unobstructed, double-glazed window to admit sunshine when the sun is out, and shutters or doors of some kind to block the opening at all other times. Other methods try to be half-window and half-wall, and are thereby, half-effective, although of some apparent use in certain climates.

The prior art as exemplified by U.S. Pat. Nos. 4,324,226, 4,046,133, 3,960,131, 3,832,992, and 4,068,652 is generally illustrative of various solar energy conversion devices utilizing a lens and/or cylindrical tubing while totally omitting use of exterior panel surface coatings for concentrating, collecting and storing energy from the sun's rays. While such devices are generally acceptable for their intended purpose, they have not proven to be entirely satisfactory in that they are complex and expensive to manufacture, bulky and inconvenient to use, functional only in limited areas of the world, exhibit complicated control mechanisms, require unusual skill and knowledge to operate, have not been proven adaptable or feasible (engineering wise or economically) to small scale mass produced consumer applications, and typically can only be utilized after substantial investment.

For example, in examining the spirit of U.S. Pat. No. 4,324,226 to Beck, contrary to a central component of the present invention, Beck discloses a method and means for using glass panes merely as a protective housing or barrier to outside weather elements, with no functionality mentioned, designed or proscribed for using the optical qualities of glass panes to optimize performance (i.e., to increase absorption and/or lower emissivity) in Beck's solar apparatus. In addition, Beck uses no exterior panel surface coatings for absorption, while the present invention uses exterior panel surface coatings for both absorption and as a sealant to retain heated air and impregnated fluids in the substrate. Also, Beck discloses a method and means for collecting and storing solar energy through use of cylindrical tubes that would prove bulky, inhibit performance, and prove cost prohibitive in applications using the present invention. Finally, Beck discloses a method and means to transfer solar energy through a series of rotating panels and gears that would prove bulky, inhibit performance, and prove cost prohibitive in applications using the present invention.

U.S. Pat. No. 4,046,133 to Cook discloses the use of a secondary inner sheathing panel to effect thermal transfer rates and makes no mention of using exterior panel surface coatings for absorption, while the present invention uses exterior panel surface coatings for both absorption and as a sealant to retain heated air and impregnated fluids in the substrate. As materials engineering has greatly improved since Cook's invention some 28 years prior to the present invention, coatings used in the present invention greatly combat excessive thermal transfer rates. Also, Cook converts thermal mass to radiant heat as a terminal achievement. The present invention converts thermal mass to radiant heat, and then uses the radiant heat to effect liquid evaporation and subsequent vapor diffusion by means of natural air flow or by means of electrical motor driven by energy supplied by conversion of previously stored solar thermal mass.

U.S. Pat. No. 3,960,131 to Angilletta, much like Trombe, et al (U.S. Pat. No. 3,832,992), discloses a method and means to collect solar energy from direct sunlight into a darkened surface placed inside a window. Angilletta and Trombe, et al do so with out aid of exterior panel surface coatings for absorption, while the present invention uses exterior panel surface coatings for both absorption and as a sealant to retain heated air and impregnated fluids in the substrate. Also, Angilletta and Trombe, et al, convert thermal mass to radiant heat as a terminal achievement. The present invention converts thermal mass to radiant heat, and then uses the radiant heat to effect liquid evaporation and subsequent vapor diffusion by means of natural air flow or by means of electrical motor driven by energy supplied by conversion of previously stored solar thermal mass.

U.S. Pat. No. 4,068,652 to Worthington discloses a method and means of collecting and storing solar energy through use of metallic substrates, and manifolds, none of which are needed or used in the embodiment of the present invention. Also, Worthington discloses a method and means to reflect condensation, which is not needed or used in the embodiment of the present invention.

As a result of the shortcomings of the prior art, typified by the above, there has developed and continues to exist a substantial need for a solar apparatus which is compact, safe, economical to operate, inexpensive, and provides efficient utilization of available solar energy. Despite this need, and the efforts of many individuals and companies to develop such devices, a safe and efficient solar apparatus has heretofore been unavailable.

### BRIEF SUMMARY OF THE INVENTION

The present invention provides a novel solar energy apparatus which is readily deployable as a diffusion apparatus for various liquids, including essential oils used in aroma therapy. The present invention provides a passive solar collection and diffusion process whereby essential oils are absorbed into a plurality of breathable membranes, that when heated by means of solar thermal mass collected by a plurality of solar thermal coatings, permit controlled breathing or diffusion. Adaptable for a set of aroma diffusion applications, the current invention improves on the 1970s vision of French scientist and inventor Felix Trombe (original developer of solar energy walls used in construction of dwellings). Through it's embodiments, the current invention advances Trombe's theory of solar thermal mass (using the latest solar coating and glazing techniques) and makes solar thermal mass economically practical on aroma diffusion applications.

Also, the apparatus of the invention is self-contained, that is, the apparatus serves both as a solar energy absorber and as a storage chamber. Thus, a separate storage device does not have to be constructed. In addition, the interior surface of the apparatus can be used for advertising space and said interior surface can functionally absorb of a plurality of standard inks and inks containing photo-chromic molecules. The solar energy apparatus of the invention is simple in design and relatively inexpensive to manufacture. Furthermore, the apparatus can be manufactured in a relatively short time. Since the solar apparatus can be installed in existing window sashes (either in automobile or dwelling glass), installation is accomplished by an automobile driver or homeowner with no formal or elaborate training or special skills required.

Disadvantages of Prior Aroma Diffusion Methods

1) Utilizes room temperature evaporation and sublimation technology that results in unevenly distributed product, 2) Rely on standard, non-renewable electricity to effect liquid evaporation, 3) odor canceling abilities of scent appear to fade after about 2 weeks, not 8-10 weeks as advertised and 4) relative to the present invention are more expensive to manufacture (in labor and materials) and are therefore less economical to the consumer, both in terms of other products' up-front purchase price and recurring energy costs to operate.

Advantages and Benefits of the Present Invention

Although a variety of delivery systems exist, (see FIG. 6) to those killed in the art, the present invention is differentiated in Performance from prior arts by the present inventions ability to provide:
High Quality Diffusion: Aroma is slowly, gently, and evenly diffused many hours after solar energy capture, even after sunset. Traditional products use air evaporation ad rapidly diffuse aromas in an uncontrolled manner, thus quickly losing their effectiveness.
Long Lasting Life: Typical cycle times to distribute aromas by means of solar heated evaporation energy are for up to 400 hours covering up to 500 cubic feet. Traditional air evaporation aroma diffusion systems distribute aromas for up to 300 hours covering up to 100 cubic feet.
Affordable: Given its long lasting life, the present invention is economical and the product uses an unlimited, free and renewable energy source—the sun.
Earth Friendly: All materials are safe for consumers to use, and utilize recycled content where possible, and the invention uses renewable energy.
Leaves Favorable Scent Impression: Research has shown that among human senses, scent has the strongest tie to memory. As a tool to imprint the olfactory senses of consumers, and hence gain consumer mind share, the present invention maintains the integrity of delicate essential oils and provide extended “nose” and “face” time among consumers, in effect advertising both the scent and brand image tied to the scent. In a novel feature, one embodiment of the invention discloses physical space on the surface of the solar evaporated air freshener to absorb a plurality of standard inks and inks containing light sensitive color changing photo-chromic molecules, with said space being used for brand, product and or service advertising.

Accordingly, it is an object of this invention to provide a relatively inexpensive room or automobile air freshener which operates safely while unattended, which can take advantage of a free and renewable source of energy, namely solar energy, thereby avoiding wasted energy, which can be easily impregnated with fragrant material, and which can be easily positioned to the inside of a window found in the typical home or automobile take advantage of and make useful otherwise latent utility of the window's ability to pass solar energy.

Other benefits and advantages of the novel solar energy apparatus of the present invention will be apparent from the above description and the accompanying drawings.

### BRIEF DESCRIPTION OF THE FIGURES

FIG. 1 is a fragmentary vertical side elevation of one form of the novel solar evaporated air freshener apparatus showing the solar energy absorption and storage of the invention;

FIG. 2 is a front interior view in section of the solar evaporated air freshener apparatus shown in FIG. 1;

FIG. 3 is a back anterior view in section of the solar evaporated air freshener apparatus shown in FIG. 1;

FIG. 4 is a top and bottom view in section of the solar evaporated air freshener apparatus shown in FIG. 1;

FIG. 5 is a view of the clip to hold the apparatus shown in FIG. 1 to louvers of an automobile ventilation system, and

FIG. 6 is an illustrative chart of the various fragrance storage and aroma diffusion technologies available, with a description of the advantages and disadvantages of each.

### DETAILED DESCRIPTION OF THE INVENTION

Summary Description

Passive solar collection and diffusion process, whereby fragranced essential oils 9 are absorbed into a plurality of breathable membranes 8, that when heated 5 and evaporated 11 by means of solar thermal mass 5 collected by a plurality of coatings 6 on the surface 5 of the apparatus, permit controlled breathing or diffusion 11 of resulting fragranced vapors. Specifically, the present invention employs a process whereby microencapsulated and non-microencapsulated essential oils 9 are impregnated into a plurality of insulating membranes 8 that permits the oils 9 to breathe 11 through the membrane surface as a vapor. The system relies on solar heated convection 5 to carry the vapor wherever air circulates by means of either passive diffusion 11 or optionally diffused by means of an automobile's ventilation system 24, with a fish hook shaped clip 25 that has a spring action arrowhead 26 to pierce the anterior side 28 and interior side 27 of the apparatus and firmly hold said apparatus in front of said louvers.

Absorption (Claim 1)

As stated in Claim 1, absorption of sun-rays 2 by the apparatus is achieved by a system of orchestrated components in the glass 3,4 and in the silicone solar coatings 6 herein. In addition, the interior surface 8 of the apparatus can be used for advertising space 13 and said interior surface can functionally absorb of a plurality of standard inks and inks containing photo-chromic molecules 6.

Glass (Claim 1)

Evaporation 11 of said plurality of microencapsulated and non-microencapsulated solids, gels, and liquids 9 into vapors is achieved by means of placing the apparatus to the inner side of conventional glass panes 3 found in buildings and automobiles (as determined by the temperance, thickness, and emissivity standards of the Uniform Building Code and the United States Department of Transportation, National Highway Traffic Safety Administration, and the Federal Motor Vehicle Safety Standards, respectively) for the apparatus to capture direct sunlight 1, with the short wave sun rays 2 being favorably amplified and long wave sun rays partially filtered by said glass 3 in buildings or automobiles.

Coating and Surface Functionality (Claim 1)

The present invention employs a plurality of highly absorptive, low emissivity solar coatings 6, 16, 19 that work in conjunction with the glass 3,4 of either an automobile or a building, and said plurality of coatings serve both as a means of solar energy collection and as a means of sealing the apparatus' outer surface for excessive heat and air loss. In one example of an individual coating 6, 16, 19 within the plurality of commercially available coatings in the embodiment of the present invention, silicon solar coatings such as Solkote Hi/Sorb-II made by Solar Energy Corporation has both optical and thermal qualities conducive to aggressively absorbing the short wave, high Ultra-Violent (UV) rays from the sun, while filtering out long wave rays and while providing low emissivity. Among other materials, Hi/Sorb-II contains dark colored powered silicone which is highly absorptive and has low-emissivity and Hi/Sorb-II contains ceramic micro-spheres 7, 20 that have thermal properties which retain and subsequently transfer absorbed solar heat to the adjacent substrate. In another embodiment, in addition to the basic silicon solar coatings 6, 16, 19, a photo-chromic 6 powder may be added to the substrate's interior surface 15 in the form of colored ink 13 for a cardboard substrate or injection extruded in the case of a polymer substrate. The addition of photo-chromic molecules 6 to the basic solar coating creates a compound with added optical properties to aid in heat transfer 5 from the apparatus anterior surface 16 to the inner porosity of its substrate 8, and to provide dynamic surface color change calibrated to changes in absorbed solar rays 3. If light is absent, the photo-chromic molecules remain at rest and are opaque in color.

In a novel feature of the invention, physical space 13 on the interior surface of the solar evaporated air freshener can be used for advertising, with physical space 13 serving as a miniature sized billboard extending, as user needs determine, to the outer most edges of the interior surface of the apparatus. The physical space 13 can absorb a plurality of standard inks and inks containing light sensitive, color changing photo-chromic molecules 7 functionally used for brand, product and or service advertising. Besides signaling to the user that the apparatus is in the process of solar energy collection, heat transfer 5, evaporation 11 and fragrance diffusion, the light sensitivity and dynamic color changing characteristics of the photo-chromic molecules 7 also serve to functionally enhance the graphic image of and draw the user's visual attention to advertisements 13 provided on interior surface of apparatus.

General Coatings Performance Metrics; Generally, the plurality of coatings 6 may be applied in a variety of methods, not limited to spray on, sub-merging in a dipping vat or chamber, brushed on, in a thickness of approximately 0.02 mm to 0.025 mm, with a coverage rate of 1 gallon covering 400 to 900 square feet, a curing dried air time of 3 days or less for 100-pound grade cardboard or polymer substrates 8. Coatings are permeable, allowing airflow to a plurality of substrates 8, can be cleaned with common solvents such as toluene or xylene, absorption and reflective optical properties that aggressively absorb short-wave UV sun rays (generally at or beyond 85% absorption for waves measured at 500 nanometer or less) and reflect long wave UV sun rays (generally at or beyond 20% reflection for waves measured at 2000 nanometers or more). Solar coatings in the present invention perform in the range of −100 degrees Fahrenheit (−73 degrees Celsius) to +1000 degrees Fahrenheit (537 degrees Celsius). Photo-chromic coatings generally perform in the range of perform in the range of—total darkness to full sunlight and in the temperature range of 5 degrees Fahrenheit (−15 degrees Celsius) to 158 degrees Fahrenheit (70 degrees Celsius).

Specific Coating Performance Metrics—In Example, Solar Energy Corp's SOLKOTE HI/SORB-II

SOLKOTE Product Description and Features

SOLKOTE HI/SORB-II is an optical coating specifically formulated for solar thermal applications. Its successful worldwide use since 1982 is based upon the following unique features:

Low cost (50-75% cheaper than black chrome)
Easy spray application (requiring very low capital investment)
Excellent high temperature tolerance (to 1000° F. [537° C.])
Excellent resistance to UV and moisture degradation
Excellent long term durability (does not lose absorptivity over time)
Excellent optical characteristics
No outgassing when correctly cured
May be used in low to high temperature glazed applications including concentrating collectors
Shipped premixed, ready to apply
Excellent shelf life (one year from date of mfg.)
Excellent coating for passive applications such as Trombe walls

Not recommended for exterior or unglazed applications

SOLKOTE PRODUCT SPECIFICATIONS

| | |
|---|---|
| Binder: | 100% silicone polymer |
| Solvent: | Xylene |
| Temperature Range: | −100° F.-+1000° F. (installed) |
| Storage Temperature: | −50° F.-80° F. (−45-27° C.) |
| Viscosity: | 25 seconds #1 Zahn's cup |
| Coverage Rate: | 400-900 square feet (40-90 square |
| | meters)/gallon depending on application |
| | equipment |
| Mixing: | Shipped pre mixed, ready for use |
| Clean Up: | Xylene, Toluene |

SOLKOTE Optical Characteristics

SOLKOTE is both thickness and substrate dependent. Ideal optical qualities for solar thermal applications are best achieved by applying a thin coat on low emissivity substrates such as aluminum, copper or stainless steel. Emissivity can range from 0.28-0.49, and absorptivity from 0.88-0.94, depending on the substrate and thickness of the dried film. Passive applications on cementitious substrates will yield high absorptivities but only moderate selectivity. Thicker applications on metallic substrates will yield high absorptivities and excellent radiation properties for radiant heating applications.

[image]

[image]

SOLKOTE Surface Preparation-Metallic Substrates

Surface preparation on metallic substrates is extremely important. Not only does a clean substrate enhance adhesion, it also lowers emissivity and improves overall optical properties. SOLKOTE exhibits excellent adhesion on many substrates with little or no surface preparation, however, the following procedures are suggested to insure the excellent optical properties and long term durability available from this product.

Degrease metallic substrates using Xylene, Toluene, Acetone or other suitable solvents. This is generally considered minimum surface preparation. Copper, aluminum and stainless steel may be lightly acid etched to remove surface oxides and lower emissivity. Aluminum may also be conversion coated to lessen future oxidation. Mild steel and galvanized surfaces should not be acid cleaned but may require priming. Use of primers will increase emissivity and may also raise absorptivity.

SOLKOTE Surface Preparation-Passive Applications

Masonry substrates for passive applications should be air cured for at least one month prior to application of SOLKOTE. Surface should be free of all paint, loose grout and dust. SOLKOTE has a natural affinity for most masonry and ceramic surfaces, but is extremely thin. Suitable primers or fillers may be used to extend coverage. SOLKOTE will yield a highly absorptive and long lived surface on cementitious and ceramic substrates, but selectivity will be limited.

SOLKOTE Applications

SOLKOTE has been formulated specifically for air atomization spray application. Simple spray guns normally used in automotive body repair facilities have proven to be quite adequate for application. Electrostatic and HVLP equipment is also excellent but substantially more expensive. Gun pressures should be kept fairly low to lessen overspray and allow good thickness control. Remote pressure supply pots should have air driven agitators and the coating should be mixed as often as is practical during application. A wet film thickness of 0.8-1.0 mils (0.02-0.025 mm) is ideal and may be easily measured using a wet film thickness gauge during application. The coating should just cover the substrate and when dry, it may be possible to barely see the substrate through the dry film. Airless application equipment is not recommended and substrate temperatures should not exceed 90° F. (32° C.) during application. Good ventilation and operator protection is imperative.

SOLKOTE Drying and Curing

Curing is highly dependent upon substrate type and ambient temperature. Skin forms within 2-5 minutes; coated absorber may normally be handled after 1-3 hours drying at room temperature. SOLKOTE will naturally cure, to a point where no outgassing will occur, within 3 days at room temperature of 60° F. (16° C.) or above. Curing may be easily accelerated by baking the coated absorber panel at temperatures ranging from 225° F. (107° C.) to 450° F. (232° C.) for a period of 15 minutes to one hour. Copper should not be cured at temperatures above 400° F. (204° C.) as it will oxidize and cause a decrease in coating adhesion. However, other metals, such as aluminum and stainless steel may be cured at temperatures up to 450° F. (232° C.). Coated absorber plates may also be placed in bright sunlight to accelerate curing. Experimentation will determine the best curing procedures for your particular environment.

Insulation (Claim 1)

Acting as the apparatus' substrate 8 to host silicone solar coatings 6 containing ceramic micro-spheres 7 and photo-chromic molecules 7 on the outer anterior surface 16 and hosting microencapsulated and non-microencapsulated fragranced solids, gels, and liquids 9 on the interior surface is a plurality of insulating materials [6] including, but not limited to, 100 pound weight cardboard or polymers sheets.

Sealing (Claim 1)

The present invention employs a plurality of highly absorptive, low emissivity solar coatings 6 that work in conjunction with the glass 3,4 of either an automobile or a building, and said plurality of coatings 6 serve both as a means of solar energy collection and as a means of sealing the apparatus' outer surface for excessive heat and air loss.

Impregnation of Microencapsulated and Non-Microencapsulated Solids, Gels, and Liquids (Claim 1)

The present invention relates to a plurality of breathable insulating substrate 8 membranes impregnated with a microencapsulated and non-microencapsulated fragranced solids, gels, and liquids 9. More particularly, the present invention relates to the infusion of the microencapsulated and non-microencapsulated fragranced solids, gels, and liquids 9 into the porosity of a plurality of breathable insulating substrate 8 membranes subsequent to mechanical application of said solar coatings 6 to reverse surface of said substrate 8.

Solar Energy Transfer (Claim 1)

The main solar energy transfer 5 in the present invention is the communication 5 of stored solar heat, in the form of thermal mass, from the breathable insulated substrate 8 to impregnated microencapsulated and non-microencapsulated fragranced solids, gels, and liquids 9, causing said impregnated microencapsulated and non-microencapsulated fragranced solids, gels, and liquids 9 to evaporate into the surrounding air.

Vapor Diffusion (Claims 2 and 3)

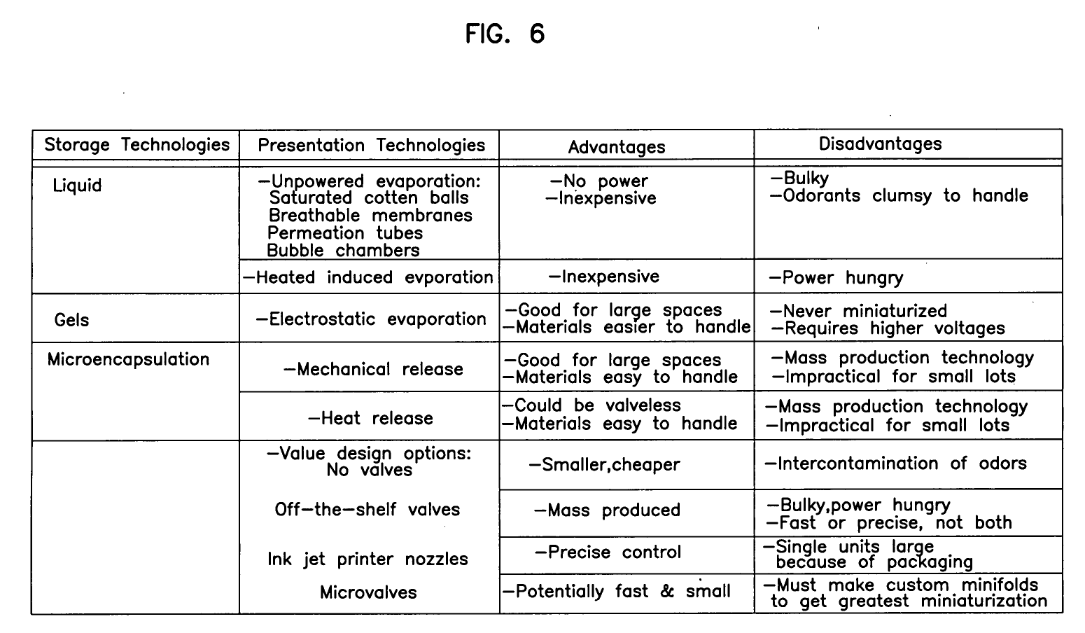

As shown in FIG. 6 ‘Olfactory Delivery Technologies’, there are a variety of vapor diffusion techniques, with a variety of corresponding advantages and disadvantages. FIG. 6 illustrates that while liquid fragrances are commonly evaporated through both powered heat induction and by un-powered means in devices such as saturated cotton balls, breathable membranes, permeation tubes, and bubble chambers, gel fragrances are commonly evaporated or sublimated by means of electrostatic charges from electrical device A third type of fragrance storage technology, (the first two being liquids 9 and gels) micro-encapsulation 9 of liquids, solids or gels achieves evaporation through mechanical means, heat release, or through pressure release from valves. In choosing between these storage technologies trade-offs in terms of performance, cost and ease of production vary significantly. Beginning with liquids 9, un-powered evaporation is inexpensive to make and operate, but they diffuse fragrances in a non-uniform manner, often releasing a burst of scent at the onset and quickly in 3 to 5 days losing their effectiveness to negate malodors. If made in larger sizes to provide additional strength and extended life, they also can be bulky and clumsy to handle. In the case of gels, electrostatic evaporation is good for large spaces, but they require continuous feed of higher voltages. As for micro-encapsulation 9 materials as used in the present invention, they can be mass-produced to create significant cost economies of scale to lower unit cost to consumer and quality of diffusion is among the most uniform due to the store and release of scent over time in multiple cells. The conclusion is that with heat-induced evaporation of liquids, gels and microencapsulated materials 9, scent is more evenly diffused, but the method can be power hungry, although with solar power at least the energy source is renewable and naturally abundant.

### DETAILED DESCRIPTION OF PREFERRED EMBODIMENTS OF THE INVENTION

Absorption (Claim 1)

As stated in Claim 1, absorption of sun-rays 2 by the apparatus is achieved by a system of orchestrated components in the glass 3,4 and in the silicone solar coatings 6 herein. In addition, the interior surface 8 of the apparatus can be used for advertising space 13 and said interior surface can functionally absorb of a plurality of standard inks and inks containing photo-chromic molecules 6.

Glass: (Claim 1)

In an exemplary embodiment, the present invention works in conjunction with glazing 3,4 standards set forth by governmental regulatory agencies such as Federal Motor Vehicle Safety Standard No. 205—stipulates Glazing 3,4 Materials—Passenger Cars, Multipurpose Passenger Vehicles, Trucks, Buses, Motorcycles, Slide-In Campers, and Pickup Covers [designed to carry persons while in motion] (Effective 1-1-68). This standard specifies requirements for glazing 3,4 materials for use in motor vehicles and motor vehicle equipment for the purpose of reducing injuries resulting from impact to glazing 3,4 surfaces. The purpose of this standard is to ensure a necessary degree of transparency in motor vehicle windows for driver visibility, and to minimize the possibility of occupants being thrown through the vehicle windows in collisions.

Coatings and Surface Functionality (Claim 1)

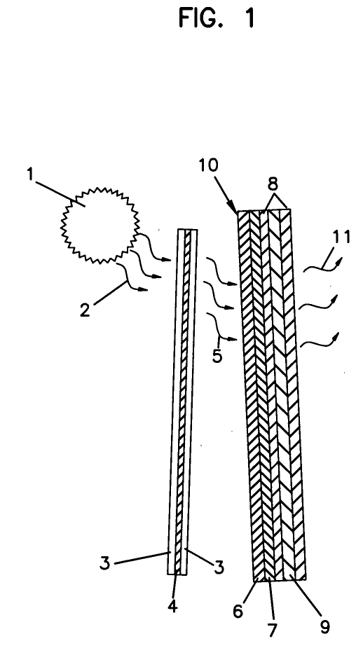

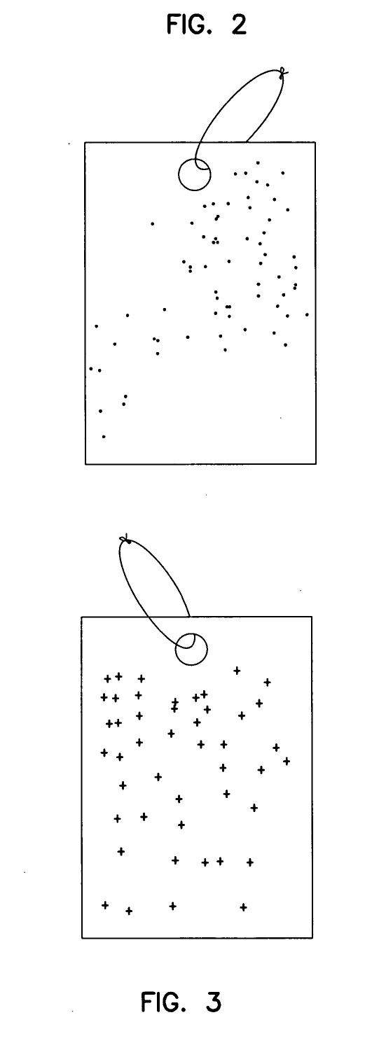

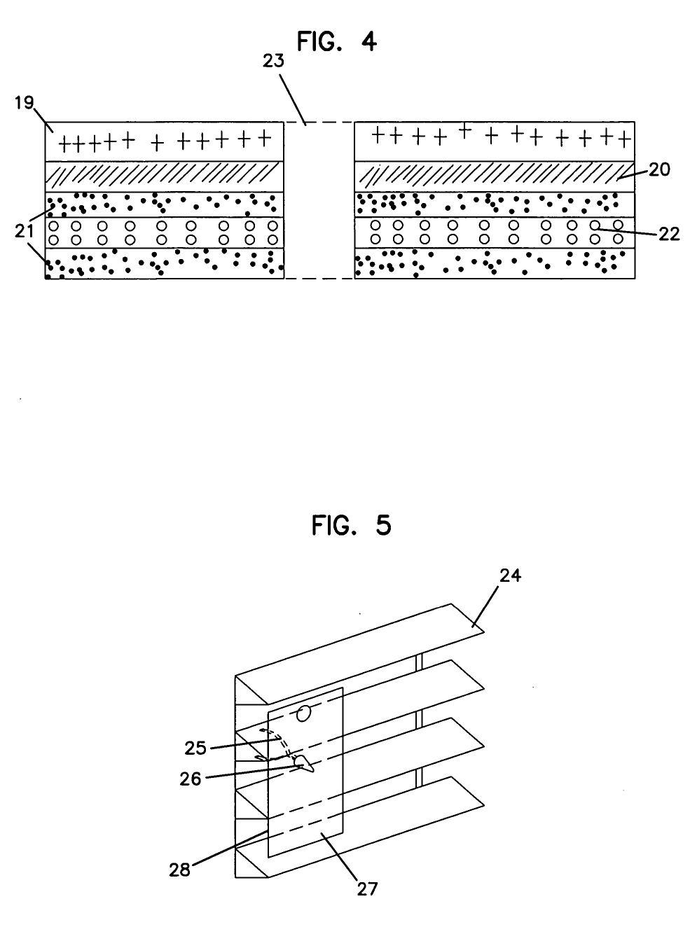

In an exemplary embodiment, as stated in Claim 1, when the sun 1 simultaneously emits long and short wave UV sun rays 2, the glass 3, and 4 of either an automobile or building contains glazing 3 and 4 films that partially filter long waves and pass through unobstructed short waves. As shown in FIG. 1, the apparatus, being positioned inside the glass 3 and 4 either in one embodiment in a hanging position (inside a building or an automobile) or in a second embodiment inserted in the vent 24 of an automobile (see FIG. 5) and attached to said vent via clips 25 and 26. As the sun rays 2 pass through the glass 3 and 4 the silicon solar coatings 6 residing on the outer anterior surface 16 of the substrate 8 aggressively absorb the short wave, high UV rays from the sun, while filtering out long wave rays and while providing low emissivity. Subsequent to sunlight light being absorbed by the silicon solar coating, the solar coating transfer 5s heated air to the ceramic micro-spheres 7 contained within the silicon solar coating. These ceramic micro-spheres 7 are arranged in a random, non-contiguous fashion so as to make a random secondary transfer 5 of heated air to various random regions of the adjoining outer anterior surface 16 of the substrate 8.

At the same time that the silicon solar coating initially absorbs sunlight, the photo-chromic molecules 6 absorb sunlight. The photo-chromic molecules are optionally contained within inks used in advertising space 13 provided on the interior substrate 8 surface or said photo-chromic molecules may be contained inside of the silicon solar coating residing on the anterior substrate 8 surface. If light is not present, the photo-chromic molecules remain at rest and are opaque in color. When light is present, optical properties of the surface bound photo-chromic molecules 6 cause dynamic changes in molecule color in stages that match the intensity of light, in general progressing in the basic five colors of the spectrum beyond opaque; first to yellow, then to red, followed by green, blue, and then black thereby signaling to the user that the process of solar energy collection, heat transfer 5, evaporation 11 and fragrance diffusion are on-going.

As stated in Claim 1, in a novel feature of the invention, physical space 13 on the interior surface of the solar evaporated air freshener can be used for advertising, with physical space 13 serving as a miniature sized billboard extending, as user needs determine, to the outer most edges of the interior surface of the apparatus. The physical space 13 can absorb a plurality of standard inks and inks containing light sensitive, color changing photo-chromic molecules 6 functionally used for brand, product and or service advertising. Besides signaling to the user that the apparatus is in the process of solar energy collection, heat transfer 5, evaporation 11 and fragrance diffusion, the light sensitivity and dynamic color changing characteristics of the photo-chromic molecules 6 also serve to functionally enhance the graphic image of and draw the user's visual attention to advertisements 13 provided on interior surface of apparatus.

Insulation (Claim 1)

In an exemplary embodiment, the plurality of insulating materials are breathable membranes that permit the reception of heated air on their outer anterior surface 16 communicated from hosted silicone solar coatings 6 containing ceramic micro-spheres 7 on said outer anterior surface 16. In addition to receiving heat transfer 5 on their outer anterior surface 16 from resident solar coatings 6, substrates 8 serve as host to impregnated microencapsulated and non-microencapsulated fragranced solids, gels, and liquids 9 on their interior surface 15. As heat is communicated, heated air is transferred 5 from the outer anterior 16 substrate surface 8, the middle portion of the substrate 8 has insulating qualities which serve to store and then later release said stored heated air to the substrate's 8 interior surface 15, whereby said liberated heated air comes into direct contact with microencapsulated and non-microencapsulated fragranced solids, gels, and liquids 9 residing on the substrate's 8 interior surface 15.

Sealing (Claim 1)

In an exemplary embodiment, as said solar coatings 6 absorb sunlight, the silicon in the coating expands to absorb solar heat. During this expansion, the silicon forms contiguous regions on the apparatus' outer anterior surface 16 to reduce heat loss back into the surrounding atmosphere. Micro-encapsulation 9 is another form of sealant taken advantage of by the present invention in that fragranced solids, gels, liquids 9 are electrostatically sealed inside of heat sensitive cellulose material. The present invention provides a method of sealing a substrate 8 sheet by providing access for fluid communication 5 of a sealant to these internal pores. The fluid communication 5 permits the flow of a sealant into the porosity of the substrate 8 sheet. Upon curing the sealant, a substantially gas impermeable anterior surface of the substrate 8 is obtained. Such a sealed sheet is useful as a heat insulating member that also host impregnated microencapsulated and non-microencapsulated fragranced solids, gels, and liquids 9

Impregnation of Microencapsulated and Non-Microencapsulated Solids, Gels, and Liquids (Claim 1)

In an exemplary embodiment, breathable insulating substrate 8 membranes that are formed from exfoliated particles have greater internal porosity or a greater amount of pores than naturally occurring substrate 8 material. Although the present invention is described as a air freshener on a flat substrate 8, other forms are suitable with the present invention. For example, exfoliated particles may be suitably processed into other shapes, such as, but not limited to, a cone, a cube, a cylinder, a disk, a prism, a parallelepiped, a sphere, and the like. As the substrate 8 sheet is formed into a denser product, the internal pores are typically inaccessible because of increased surface layers and constituent layers of carbon atoms below the surface that are substantially parallel to that surface of the sheet. These layers block the flow of coatings 6 into the pores of the substrate 8 sheet. Microencapsulated and non-microencapsulated fragranced solids, gels, and liquids 9 will, however, flow readily within the substrate 8 sheet in directions parallel to opposed parallel planar surfaces if the sealant can first penetrate past the parallel surface atoms.

Solar Energy Transfer (Claim 1)

In an exemplary embodiment, heat transfer 5 is conducted with a plurality of insulating materials which are breathable membranes that permit the reception of heated air on their outer anterior surface 16 communicated from hosted silicone solar coatings 6 containing ceramic micro-spheres 7 on said outer anterior surface 16. In addition to receiving heat transfer 5 on their outer anterior surface 16 from resident solar coatings 6, substrate 8s serve as host to impregnated microencapsulated and non-microencapsulated fragranced solids, gels, and liquids 9 on their interior surface. As heat is communicated heated air is transferred 5 from the outer anterior substrate 8 surface, the middle portion of the substrate 8 has insulating qualities which serve to store and then later release said stored heated air to the substrate's 8 interior surface, whereby said liberated heated air comes into direct contact with microencapsulated and non-microencapsulated fragranced solids, gels, and liquids 9 residing on the substrate 8's interior surface 15.

Vapor Diffusion (Claims 2 and 3)

In an exemplary embodiment as stated in claim 2, the apparatus for absorbing and transfer 5ring solar energy according to claim 1 wherein said means for affecting vapor movement is achieved by natural air flow. In using micro-encapsulation 9 as a storage technology, the present invention transfers 5 stored heat from ceramic micro-spheres 7 coated on the outer anterior surface 16 of the substrate 8 onto the interior side 15 of the substrate 8, thereby causing the heated interior substrate 15 surface to effect a secondary transfer 5 of heat to neighboring micro-encapsulated fragrance cells 9 residing on the interior substrate 8 surface. Because the micro-encapsulated fragrance cells are randomly placed on the interior surface of the substrate 8, and because heat dispersion from the ceramic micro-spheres 7 contained in the solar coatings 6 is randomly transferred 5 to various locations of the anterior surface 16 of the substrate 8, which in turn transfers 5 heat to matching locations of the reverse interior surface of the substrate 8, micro-encapsulated fragrance cells 9 are randomly heated, therefore providing for gradual progressive heating of regions of the micro-encapsulated fragrance cells over a period of time estimated in months, instead of simultaneously heating all regions containing micro-encapsulated cells. In the present invention, lateral surface temperature transfers 5 between regions of micro-encapsulated fragrance cell is controlled by the random, rather than contiguous placement of heat absorbent ceramic micro-spheres 7 in the outer anterior surface 16 solar coating. As the surface temperature of each affected micro-encapsulated cell rises from the transfer 5 of heat from the adjoining substrate 8, the micro-encapsulated cell walls begin to fatigue and degrade, simultaneously collapsing and transferring 5 heat inwardly and liberating the previously encapsulated solids, gels, or liquids 9. Upon immediate release from the micro-encapsulated cell walls, the heated solids, gels, and liquids 9 begin evaporation 11 into the surrounding ambient air, with the evaporation being accelerated by the solar heated condition imposed upon them prior to their release from the micro-encapsulated cell walls.

In an alternative embodiment as stated in claim 3, the apparatus for absorbing and transferring 5 solar energy according to claim 1 wherein said means for affecting vapor movement is achieved by connecting the present invention, with its attachable clip, to the louvers of an automobile's ventilation system 24 that, when blowing air, diffuses fragrance into the air inside of an automobile. Again, in using micro-encapsulation 9 as a storage technology, the present invention transfers 5 stored heat from the ceramic micro-spheres 7 coated on the outer anterior surface 16 of the substrate 8 onto the interior side 15 of the substrate 8, thereby causing the heated interior substrate 8 surface to effect a secondary transfer 5 of heat to neighboring micro-encapsulated fragrance cells 9 residing on the interior substrate 15 surface. Because the micro-encapsulated fragrance cells are randomly placed on the interior surface of the substrate 8, and because heat dispersion from the ceramic micro-spheres 7 contained in the solar coatings 6 is randomly transferred 5 to various locations of the anterior surface 16 of the substrate 8, which in turn transfers 5 heat to matching locations of the reverse interior surface 15 of the substrate 8, micro-encapsulated fragrance cells 9 are randomly heated, therefore providing for gradual progressive heating of regions of the micro-encapsulated fragrance cells over a period of time estimated in months, instead of simultaneously heating all regions containing micro-encapsulated cells. In the present invention, lateral surface temperature transfers 5 between regions of micro-encapsulated fragrance cell 9 is controlled by the random, rather than contiguous placement of heat absorbent ceramic micro-spheres 7 in the outer anterior surface 16 solar coating. As the surface temperature of each affected micro-encapsulated cell rises from the transfer 5 of heat from the adjoining substrate 8, the micro-encapsulated cell walls 9 begin to fatigue and degrade, simultaneously collapsing and transferring 5 heat inwardly and liberating the previously encapsulated solids, gels, or liquids 9. Upon immediate release from the micro-encapsulated cell walls, the heated solids, gels, and liquids 9 begin evaporation into the surrounding ambient air, with the evaporation being accelerated by the solar heated condition imposed upon them prior to their release from the micro-encapsulated cell walls, and secondarily with the evaporation being further accelerated by the heated air from the automobile ventilation system 24.

Having thus described the preferred embodiments in some detail it will be clear to those having ordinary skill in the art that obvious modifications could be made to the system without departing from the spirit of the invention. Such obvious modifications are within the inventive concept. It will be apparent that various modifications can be made in the particular solar energy apparatus described in detail above and shown in the drawings within the scope of the invention. For example, the size and configuration of the components can be changed to meet specific requirements. Also, the solar energy absorbing members can be disposed in various orientations provided the operation and functioning of the apparatus are not deleteriously affected. In addition, the solar energy absorbing material can be arranged differently in the absorbing members. Various changes may be made in shape, size and arrangement of parts. For example, equivalent elements or materials may be substituted for those illustrated and described herein. Parts may be reversed, and certain features of the invention may be utilized independently of the use of other features, all as would be apparent to one skilled in the art after having the benefit of having this description of the invention. It is not intended that the words used to describe the invention nor the drawings be limiting on the invention, rather the only limitations placed are those in the appended claims.

1. An air freshener apparatus delivering passive solar collection, solar energy transfer, evaporation of a plurality of solids, gels, and liquids, vapor diffusion process, and consisting of a panel for absorbing solar energy, a process for coating said panel with a plurality of highly solar heat absorptive materials and optional photo-chromic coatings, a plurality of breathable insulating substrate membranes directly adjacent to said coated panel, a sealant, impregnation of a plurality of microencapsulated and non-microencapsulated solids, gels, or liquids into plurality of breathable insulating substrate membranes, primary solar energy transfer from said solar coated surface to a plurality of breathable insulating membranes to achieve thermal radiation of ambient air for convective heat-induced evaporation of said microencapsulated and non-microencapsulated solids, gels, and liquids into vapors. Solar filtering, collection, and subsequent evaporation of said plurality of microencapsulated and non-microencapsulated solids, gels or liquids into vapors is achieved by means of placing the apparatus to the inner side of a plurality of standard glass panes found in automobiles and buildings (standard as defined by the temperance, thickness, and emissivity standards of the the United States Department of Transportation, National Highway Traffic Safety Administration, and the Federal Motor Vehicle Safety Standards, and the Uniform Building Code, respectively) for the apparatus to capture sunlight that has been treated by first passing through said glass panes of automobiles and buildings which have favorably amplified said sunlight's short wave ultra violet (UV) sun rays and favorably filtered said sunlight's long wave UV sun rays. Secondary solar energy transfer from said plurality of solar coatings on apparatus surface to a plurality of breathable insulating membranes is used to achieve diffusion of said vapors and associated fragranced aromas. In a novel feature, one embodiment of the invention discloses physical space on the surface of the solar evaporated air freshener to absorb a plurality of standard inks and inks containing light sensitive color changing photo-chromic molecules with said surface space being used for brand, product and or service advertising.

2. An air freshener apparatus according to claim 1 wherein said means for affecting vapor movement is achieved by natural air flow.

3. An air freshener apparatus according to claim 1 wherein said means for affecting vapor movement is achieved by connecting the present invention, with its attachable clip, to the louvers of an automobile's ventilation system that, when blowing air, diffuses fragrance into the air inside of an automobile.
